(19) United States
(12) Patent Application Publication
(10) Pub. No.: US 2010/0087831 A1
(43) Pub. Date: Apr. 8, 2010

(54) KNEE REPLACEMENT NAIL REMOVER

(76) Inventor: Donald Marx, Plainview, NY (US)

Correspondence Address: LACKENBACH SIEGEL, LLP, LACKENBACH SIEGEL BUILDING, 1 CHASE ROAD, SCARSDALE, NY, 10583, US

(21) Appl. No.: 12/246,766

(22) Filed: Oct. 7, 2008

(51) Int. Cl. A61B 17/58

(52) U.S. Cl. 606/104

(57) ABSTRACT

An orthopedic fixation pin removal device includes a lever rotatively disposed on a body, the lever is operably disposed by a rack and pinion, with pivoting of the lever the rack is moved in unison with a fixation pin engaging assembly, so that the fixation pin is moved in uniform motion. A fixed grip is oppositely disposed from the lever to provide bracing during removal of the fixation pin.

### BACKGROUND OF THE INVENTION

1. Field of the Invention

This invention relates to a tool employed in orthopedic fixation procedures, and more specifically, to the withdrawal and extraction of orthopedic fixation pins from the bone upon completion of a surgical procedure. The invention relates to such tools in knee replacement surgical procedures. The invention also relates to improvements in surgical methods and procedures using such tools.

2. Background and Discussion of Related and Unrelated Art

Orthopedic surgery requires the use of template, measuring, cutting and other devices temporarily affixed to the bones. These devices can be affixed using a variety of methods, one of which is the use of pins with heads. Once the surgeon is finished with the device, the pins must be removed. The pins, which are driven in a manner similar to nails, are firmly held in the bone. It is most important that pins be removed using a force applied parallel to the longitudinal axis of the pin without generating lateral forces. Removing the pins using a claw hammer type of remover would damage the pins, the device, and the underlying bone. Equipping the pins with heads offers a gripping surface to aid in pin removal. Prior art pin removers are generally like pliers or pullers, gripping the head of the pin in the jaws while force is applied to the jaws by the handles of the pin puller. The prior art pin pullers require the user to pull the pin out using the strength of the user. Often, when the frictional force is overcome, the pin moves somewhat freely or jiggles sideways allowing the user's hand and arm to move in an uncontrolled manner. This sideways or lateral movement may cause undesired movement or displacement of the prosthetic implant.

Typical prior art pliers surgical pin extraction tools or pullers are disclosed in U.S. Pat. No. 7,189,243, to Seelig et al. and U.S. Pat. No. 6,066,143 to Lane. Such pliers type surgical pin extraction tools are in general use in orthopedic fixation pins withdraw procedures. The surgeon in squeezing and pulling the pliers would likely cause undesired lateral movements.

One attempt to improve the removal of surgical pins is disclosed in US 2006/0200159 to Collazo et al. Collazo et al. discloses a tibia alignment guide assembly and a fixation or anchor pin is adapted to anchor the guide. An extraction lever is actuated to remove the pins.

Another attempt to improve the extraction of a pin from a bone is disclosed in US 2005/0149051 to Hansson. The Hansson tool is in the form of a tee, having a tubular receiving section and an extraction member with a tip which screws into the pin, which pin is drawn into the tube.

Another attempt is disclosed in U.S. Pat. No. 6,673,078 to Muncie. Muncie discloses a pair of flexible jaws to grip the surgical wires or pins

Still another attempt is disclosed in U.S. Pat. No. 6,066,143 to Lane. Lane discloses a pistol grip and squeezing handle with a lever action to pull the pin from the bone.

The orthopedic pin extraction art tools suffered from one or more of the following impediments; the need for and creation of lateral forces by the requisite jiggling action of the user, which lateral action caused displacement of the orthopedic device in the removal of the pin; an elaborate mechanical construction which was not practical or suitable for surgical operating procedures and/or a too complex construction that was difficult to manufacture and operate without causing adverse effects on the successful implantation particularly in knee replacement procedures.

The present invention provides an improved means for removing orthopedic pins from a bone, particularly knee replacement procedures, while advoiding undesired lateral forces. The present invention also provides a tool as aforesaid, is of a practical design and construction and readily operable in surgical procedures.

The unrelated art directed to industrial nail removal provides various nail extraction tools such as disclosed in U.S. Pat. No. 5,989,272 to Crider, U.S. Pat. No. 6,136,004 to Keller and U.S. Pat. No. 6,755,392 to Phillips.

The unrelated art directed to cork extraction for wine bottles discloses a winged corkscrew, generally referred to as a “rabbit ears” construction. The winged corkscrew construction is disclosed in U.S. Pat. No. 6,823,760 to Kilduff, which reference is incorporated herein in its entirety by reference thereto.

### SUMMARY OF THE INVENTION

This invention is an orthopedic pin extractor wherein the extraction forces are readily generated and coaxial with the pin and essentially free of undesired lateral forces which could cause adverse displacement of the prosthetic. The orthopedic pin extractor includes a rack and pinion construction with the rack centerline being in parallel disposition with the centerline of the pin end engaging parallel component which has a centerline in alignment with the orthopedic pin. The orthopedic pin extractor of the present invention includes a claw or grip component for releasable engaging the proximate end of a straight pin, and a lever pivotally connected to a rack and pinion, and co-aligned with a bearing arm that holds the extractor in place, and the lever is rotated to actuate the rack and pinion and in turn to cause a proximate directional force to be exerted on the pin.

### BRIEF DESCRIPTION OF THE DRAWINGS

FIG. 1 is a side elevation view of the device prior to engagement with the proximate end of a headed orthopedic pin;

FIG. 2 is a side elevational partial fragmentary view of the device of FIG. 1 after removal of the orthopedic pin;

FIG. 3 is a top plan view of the device of FIG. 1;

FIG. 4 is a partial sectional side elevational view of the device of FIG. 1 showing initial engagement of the orthopedic pin;

FIG. 5 is a partial sectional side elevational view of the device as shown in FIG. 4 with the orthopedic pin fully withdrawn;

FIG. 6 is a partial sectional side elevational view of an alternate embodiment showing initial engagement with a headless orthopedic pin;

FIG. 6A is an enlarged fragmentary view of FIG. 6; and

FIG. 7 is a partial sectional side elevational view of the embodiment of FIG. 6 with the headless orthopedic pin fully removed.

### DESCRIPTION OF THE INVENTION

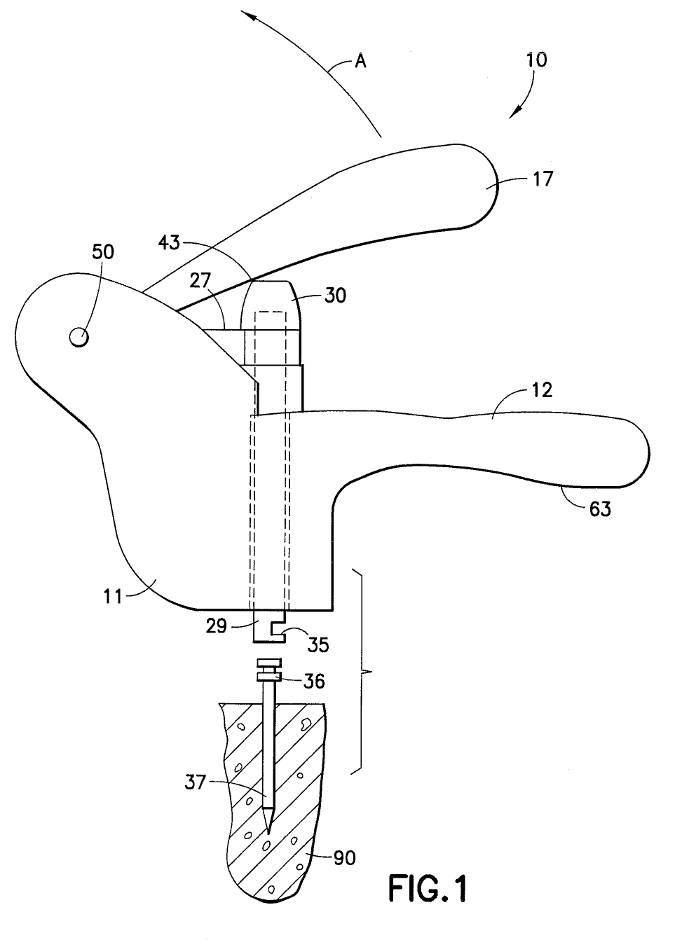

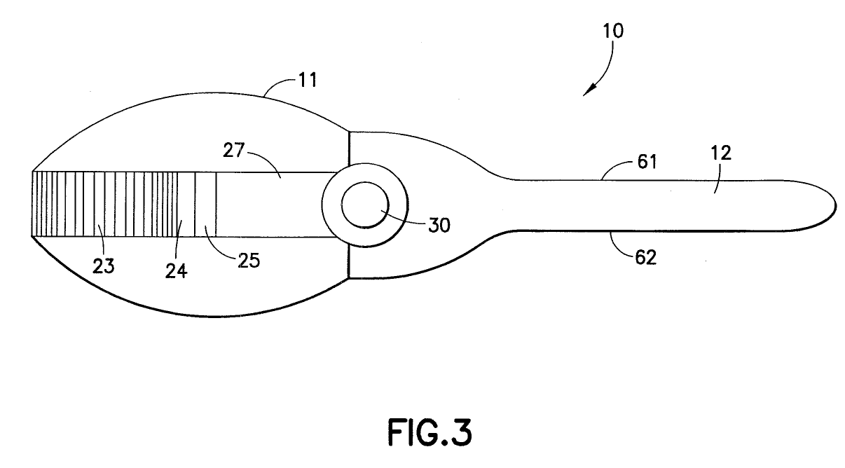

Referring to FIGS. 1-5, there is shown the knee replacement nail removal or orthopedic fixation pin removal device 10 of the present invention. Device 10, in general, includes a body 11, a grip 12 formed with or fixedly disposed on body 11, a throughhole 13 extending from the proximate end 14 to the distal end 15 of body 11, pivot assembly 16, lever 17 pivotally or rotatively disposed by assembly 16 to body 11, and a nail or pin engagement assembly 20 slidably disposed in hole 13, as will be more fully explained thereinafter.

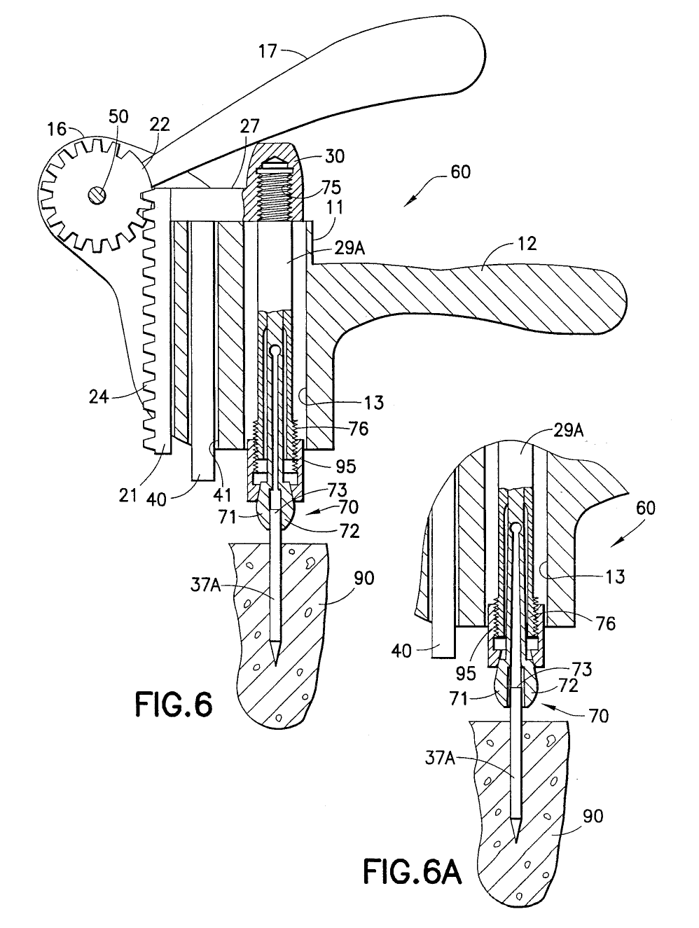

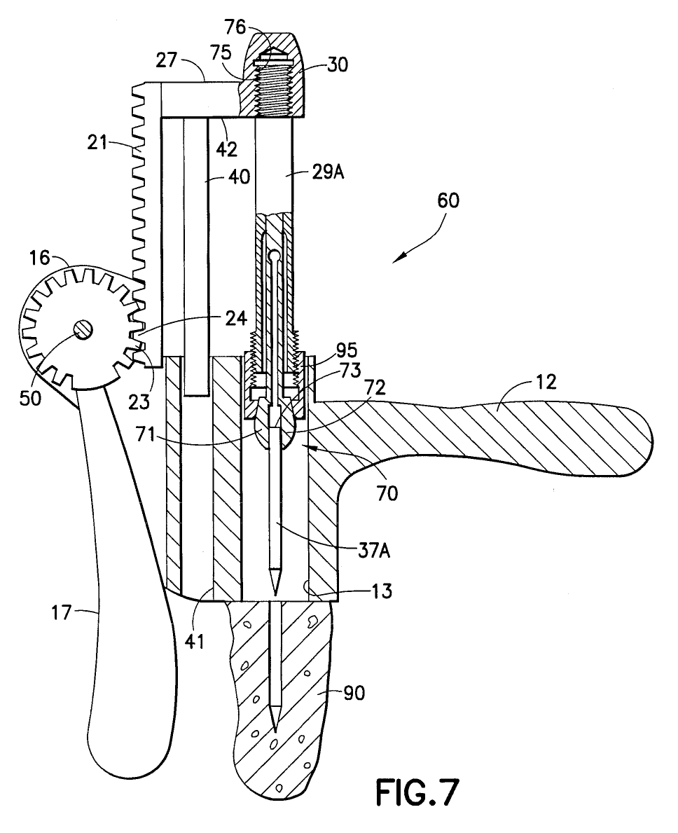

A rack 21 and gear or pinion 20 is operably disposed with respect to lever 17, assembly 16 and body 11. Lever 17 is fixedly mounted on pinion 20, as at 22. The pinion teeth 23 operably engage rack teeth 24. Rack 21 has a proximate end 25 and a distal end 26. Rack proximate end 26 is fixedly mounted to cross member 27, as at 28. Cross member 27 is fixedly mounted to cap 30. Cap 30 has screw threads for receiving the proximate end of stem or rod 29 of nail engagement assembly 16 as at 31. Cap 30 covers cross member 27 and stem end 31 and completes the distal end construction for assembly 20. Stem 29 is slidably disposed in throughole 13. The distal end 32 of stem 29 is formed with claw 35 for engaging the head 36 of nail 37. Claw 35 may be of any desired size and configuration to conform to and frictionally engage the head of a fixation pin or orthopedic nail, as will be further discussed particularly in relation to the embodiment of FIGS. 6-6A and 7.

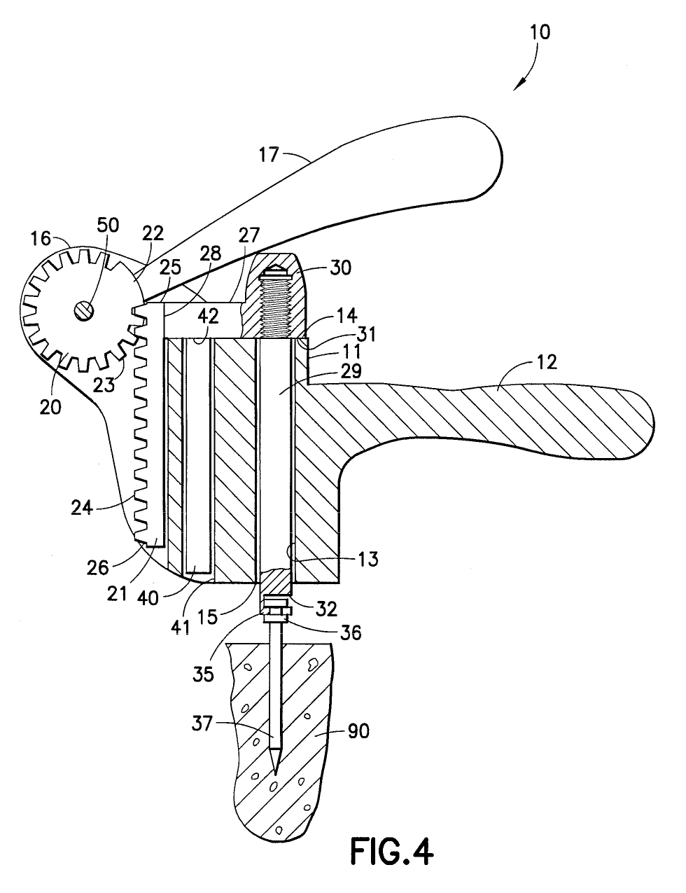

Referring specifically to FIGS. 1 and 4, lever 17 is disposed in a first position wherein stem 39 is fully withdrawn or proximately disposed in throughhole 13. Nail 37 is shown disposed in bone 90 and requires withdrawal. Nail 37 is in co-linear disposition with respect to stem 29 (FIG. 1). Nail head 36 is frictionally engaged in claw 35 (FIG. 4). A guide bar 40 is slidably disposed in hole 41 formed in body 11. Guide bar 40 is in parallel disposition with stem 29. Guide bar 40 is fixedly attached to cross member 27, as at 42. Lever 17 is prevented from further clockwise movement by stop portion 43 of cap 30. In this manner of construction, lever 17 is rotated in the counter-clockwise direction of arrow A (FIG. 1) to begin to withdraw nail 37. Nail head 36 is shown as a headed nail but other nail heads are within the contemplation of the present invention.

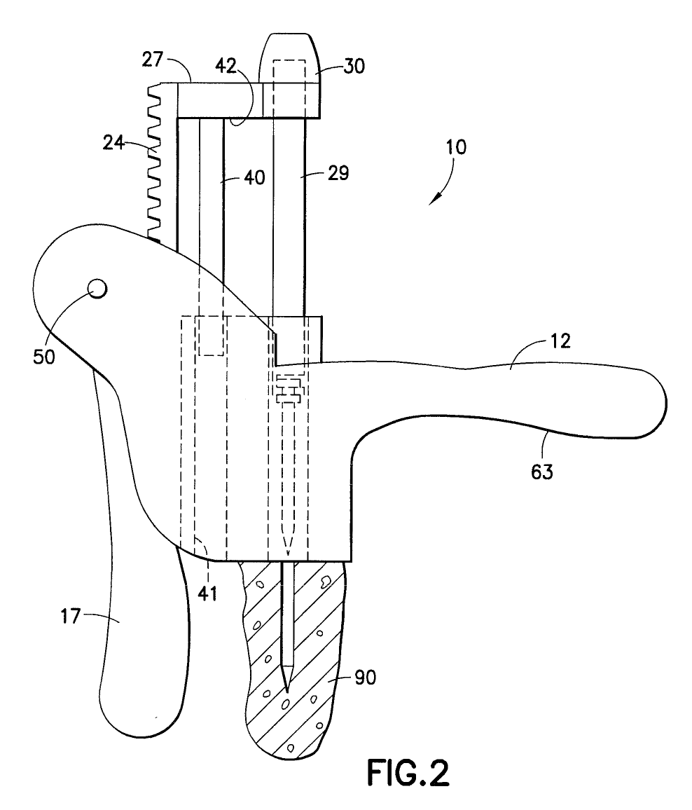

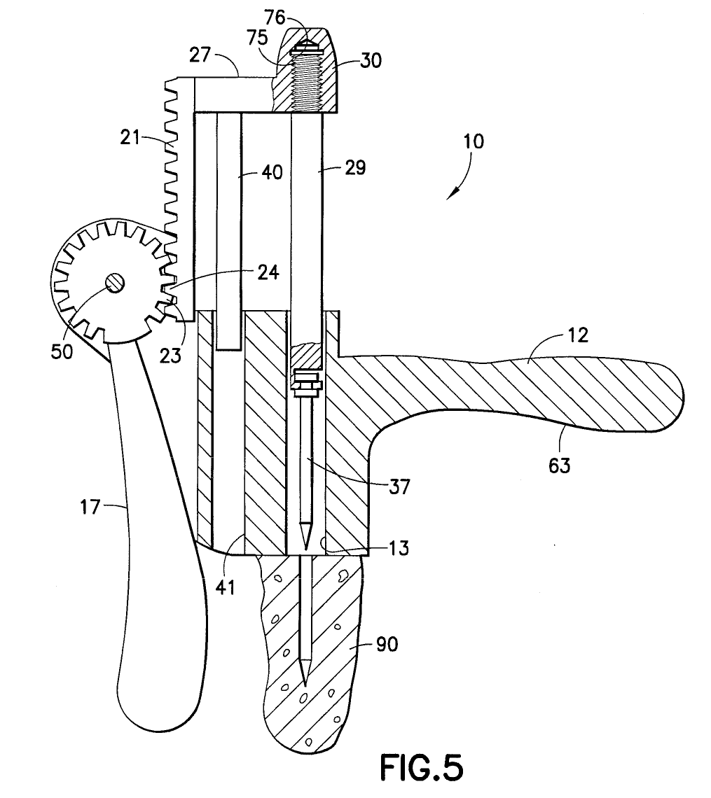

Referring to FIGS. 2 and 5, there is shown lever 17 fully pivoted in the counter-clockwise direction. Pivot assembly 16 includes pivot pin 50 having axis 51. Pinion 20 is rotatively disposed on pin 50, and lever 17 is fixedly disposed on pinion 20. Rack 21 moves in the proximate direction with the counter-clockwise rotation of lever 17 as shown by arrow A. Rack 21, cross member 27 and stem 29 and guide bar 40 move in unison.

In the aforesaid manner of construction and operation, nail 37 is moved in the proximate direction and is withdrawn from the bone, is a smooth controlled manner without undesired jiggling and without the formation of undesired lateral forces. The removed nail is readily withdrawn from the claw.

Lever 17 is shaped in a rabbit ear configuration. This permits the generation and transmission of a even force to stem or rod 29. Grip 12 is formed with generally parallel sides 61 and 62 and a contoured distal portion 63 to provide an ergonomic grip to steady body 10 during the pivoting of lever 17 and consequential removal of pin 37.

Referring to FIGS. 6-6A and 7, there is shown an alternate embodiment of the invention 60, and particularly an alternate pin engagment construction, namely assembly 70. Embodiment 60 includes construction elements, namely, body 11, grip 12, throughole 13, assembly 16 lever 17 and rack and pinion 20-21 which elements are similar in construction and operation to like elements described with respect to embodiment 10. Assembly 70 of embodiment 60 is formed with opposed flexible or elastic arms 71 and 72 for engaging the proximate end 73 of a headless orthopedic fixation pin 137 or surgical wire. The headless pin engagement construction 70 of FIGS. 6, 6A and 7 may be constructed as shown in U.S. Pat. No. 6,673,078 to Muncie, which disclosure is incorporated herein in its entirety by reference thereto.

Stems 29 and 29A of embodiments 10 and 60 respectively, are formed at the proximate end, with attachment elements, such as screw threads 75 on stem 29 and screw threads 76 on stem 29-29A for replaceable attachment of alternate nail or pin engaging constructions, such as claw 35 and altermatively elastic arms 71 and 72.

Device 10 may be constructed of surgical metal which can be readily and conveniently sterilized between operations.

While the foregoing description provides a single pivoted lever in combination with a fixed grip, it is also within the contemplation of the present invention to provide two rabbit eared levers that are in co-pivoting and co-bracing operable disposition. The two rabbit eared levers would be co-operatively and simultaneously pivoted to remove the fixation pin.

The foregoing describes certain embodiments of the invention, and is not intended to limit the invention, which invention is defined by the adjoined claims.

What is claimed is:

1. A device for the removal of an orthopedic fixation pin, said device comprising:
a body, said body having a first handle extending outwardly from the body, said body having an elongate hole,
a second handle, and means for rotatively connecting the second handle to the body;
a rod, means for engaging the proximate end of the pin, said means for engaging the proximate end of the pin being connected to said rod, said rod being slidably disposed in said hole; and
means for moving the rod and in turn the means for engaging the fixation pin, said means for moving the rod being operably connected to the second handle;
whereby the first handle is gripped, the proximate end of the fixation pin is engaged and the second handle is rotated to cause the rod to move in the proximate direction and withdraw the fixation pin.

2. The device of claim 1, said means for moving the rod comprises a rack and pinion, said second handle being connected to said pinion.

3. The device of claim 2, said first handle being fixedly disposed on said body.

4. The device of claim 2, said rack being in parallel disposition with the rod whereby rack and rod move in unison and in parallel disposition during withdrawal of the fixation pin.

5. The device of claim 1, said elongate hole comprises a centerline, said nail being disposed in co-linear alignment with the center line, said rack being disposed laterally from and in parallel disposition with the center line.

6. A device for the removal of an orthopedic fixation pin, said device comprises:
a body;
means for engaging the proximate end of an orthopedic fixation pin movably disposed in the body;
a rack and pinion assembly operably connected to the means for engaging the proximate end of the orthopedic fixation pin; and
a lever rotatively connected with respect to the body and operably connected to the pinion;
whereby the lever is rotated and the rack and in tern the means for engaging the pin moved in unison to remove the orthopedic fixation pin.

7. The device of claim 6, wherein the rack comprises a centerline, and the means for engaging the pin comprises a centerline, said centerlines being in co-linear alignment with the pin centerline.

8. The device of claim 6, said body, further comprises a grip extending outwardly from the body.

9. The device of claim 8, said lever and grip being oppositely outwardly disposed from the body.

10. The device of claim 6, said lever being operably movable in the distal direction to cause the proximate direction removal of the pin.

11. A device for the removal of an orthopedic fixation pin, said device comprises:
a body having an elongate throughhole;
means for engaging the proximate end of a fixation pin being slidably disposed in said hole;
a lever, means for rotatively connecting the lever with respect to the body;
and means for operably connecting the means for engaging the proximate end of the fixation pin with the means for rotatively connecting the lever;
whereby the lever is rotated in the distal direction to cause the fixation pin to be withdrawn in the proximate direction.

12. The device of claim 11, further comprises a grip fixedly disposed on the body, and oppositely disposed, said grip braces the device during rotation of the lever and removal of the pin.

13. The device of claim 11, said lever being configured as a rabbit ear.

14. The device of claim 13, said grip being ergonomically configured.

15. The device of claim 14, said means for rotatively connecting the lever with comprises a rack and pinion.
